(19) United States
(12) Patent Application Publication
(10) Pub. No.: US 2018/0250430 A1
(43) Pub. Date: Sep. 6, 2018

(54) AIR MONITORING AND FILTRATION DEVICE FOR DETECTING AND REMOVING ODORS

(71) Applicant: HOUND TECH LLC, Coral Gables, FL (US)

(72) Inventors: Brian L. Machovina, Coral Gables, FL (US); Eileen McHale Machovina, Coral Gables, FL (US)

(21) Appl. No.: 15/909,344

(22) Filed: Mar. 1, 2018

Related U.S. Application Data
(60) Provisional application No. 62/465,963, filed on Mar. 2, 2017.

(51) Int. Cl. A61L 9/20; A61L 9/12; B01D 46/00; B01D 46/10; B01D 46/44; B01D 53/30

(52) U.S. Cl. CPC A61L 9/20; A61L 9/122; B01D 46/0005; B01D 46/0036; B01D 46/10; B01D 46/442; B01D 2257/406; B01D 2273/30; B01D 2253/102; B01D 2253/204; B01D 2257/304; B01D 2257/7025; B01D 2257/306; B01D 53/30

(57) ABSTRACT

An odor detection and filtration device is provided including a housing enclosing a void and having and air inlet and air outlet. The void contains, and the housing encloses, one or more sensors, one or more filters, one or more fans, and a control unit. The control unit may be in electrical communication with the one or more sensors and one or more fans. The control unit may activate the one or more fans upon the one or more sensors detecting the presence of one or more odors in the ambient air surrounding the device. The one or more fans draw ambient air through the air inlet into the void and subsequently downstream through the filter and out the device through the air outlet.

### CLAIM OF PRIORITY

This application is being filed as a non-provisional patent application under 35 U.S.C. § 111(a) and 37 CFR § 1.53(b). This application claims priority under 35 U.S.C. § 119(e) to U.S. provisional patent application Ser. No. 62/465,963 filed on Mar. 2, 2017, the contents of which are incorporated herein by reference.

### FIELD OF INVENTION

The invention relates generally to air filtration devices and in particular to a device and method for the detection and filtration of odors from flatulence, defecation, or any unpleasant offensive odors in ambient air.

### BACKGROUND OF THE INVENTION

Air purifiers and air filters are widely used in interior spaces such as homes and offices to minimize the amount of dust, allergens, and micro-organisms that are present in the air. These systems typically include a fan for circulating air and a mechanical filter disposed in an air path to filter or purify air flowing therethrough.

Larger air purifiers and air filters tend to be more effective due to the large volume of air they can filter and purify at a given time. However, larger air purifiers and air filters are usually obtrusive and take up a considerable amount of space in the home or office. Further, these larger systems may require more electricity and are often loud due to the size of the fans included in the system.

Although air purifiers and air filters may provide a solution to combating the presence of contaminants such as dust, allergens and micro-organisms, they lack the means to detect unpleasant odors and subsequently remove the odors from the air. Individuals are well aware of the offensive odors that may be released from flatulence, feces, or urine. These odors can be disruptive and unpleasant to individuals located in the vicinity of such odors. When unpleasant odors are present in the air, individuals usually use candles, air freshener sprays, plug in air fresheners or the like, to “cover-up” the unpleasant odor with a more pleasant scent. However, this tactic merely masks the presence of the unpleasant odor and does nothing to actually remove the odor.

Additionally, the use of candles or air fresheners presents an unfortunate problem because an individual must first come in contact with the unpleasant odor before a candle or air freshener can be used to mask the scent. Furthermore, while the scent of candles or air fresheners may provide a more pleasant odor as compared to odors released from flatulence, feces or urine, some individuals may not enjoy the scent of candles or air fresheners.

Accordingly, the current invention aims to provide an air filtration device that may be placed in various locations to detect and remove odors resulting from flatulence, defecation, urination, or any unpleasant or offensive odors.

### SUMMARY OF THE INVENTION

The following summary is provided to introduce a selection of concepts in a simplified form that are further described below in the detailed description. This summary is not intended to identify key features or essential features of the claimed subject matter, nor is it intended to be used to limit the scope of the claimed subject matter.

The present disclosure is directed to an odor detection and filtration device that detects and removes odors resulting from flatulence, defecation, urine, or any unpleasant or offensive odor.

According to one implementation, the odor detection and filtration device includes a housing having an air inlet and an air outlet. The housing of the device is generally hollow and the interior of the housing defines an open or “void.” The void facilitates the collection of ambient air that enters the housing and defines an air passage that extends from the air inlet to the air outlet. The void contains, and the housing encloses, one or more sensors, one or more filters, one or more fans, and a control unit. Alternatively, the sensors, filters or fans may be situated outside the device.

The one or more sensors are electrical, optical, chemical or bioengineered sensors, including without limitation, MOS, Electrochemical, Electrolysis, MEMS, and Infrared sensors, that detect the presence of one or more gases or chemical compounds commonly found in odors resulting from flatulence or released during defecation and/or urination in both humans and domestic animals. The one or more filters are disposed within the void of the housing and are adapted to remove odors and particulates from ambient air that has entered the void. The one or more fans may be positioned at or near the air outlet or air inlet of the device (inside or outside the device) and adapted to draw ambient air through the air inlet into the void and subsequently downstream through the one or more filters and out the device through the air outlet.

The control unit may be in electrical communication with the one or more sensors and one or more fans. The control unit may activate the one or more fans upon the one or more sensors detecting the presence of one or more odors in the ambient air surrounding the device. In response to the one or more sensors sensing the presence of the one or more odors in the ambient air, the one or more sensors send a fan-activation signal to the control unit. In response to receiving the fan-activation signal, the control unit activates one or more fans, causing the device to draw ambient air through the air inlet into the void and subsequently downstream through the filter and out the device through the air outlet.

These and other features and advantages will be apparent from a reading of the following detailed description, and a review of the appended drawings. It is to be understood that the foregoing summary, the following detailed descriptions, and the appended drawings are only explanatory and are not restrictive of various aspects claimed.

Although the invention is illustrated and described herein as embodied in an odor detection and filtration device, it is nevertheless not intended to be limited to only the details shown, since various modifications and structural changes may be made therein without departing from the spirit of the invention and within the scope and range of equivalents of the claims.

### BRIEF DESCRIPTION OF THE DRAWINGS

FIGS. 1A-1B are front angle and rear angle views of the odor detection and filtration device in accordance with an implementation of the invention.

FIG. 2 is a frontal view of the odor detection and filtration device in accordance with an implementation of the invention.

FIG. 3 is a rear view of the odor detection and filtration device in accordance with an implementation of the invention.

FIG. 4 is a top view of the odor detection and filtration device in accordance with an implementation of the invention.

FIG. 5 is a cross section side view of the void of the housing of the odor detection and filtration device in accordance with an implementation of the invention.

FIG. 6 is a cross section side angle view of the void of the housing of the odor detection and filtration device in accordance with an implementation of the invention.

FIG. 7 is a cross section top view of the void of the housing of the odor detection and filtration device that shows the filter in accordance with an implementation of the invention.

FIG. 8A-8B are cross section top and cross section top angle views of the void of the housing of the odor detection and filtration device that shows the control unit in accordance with an implementation of the invention.

FIG. 9 is an illustration of an exemplary computing environment in accordance with an implementation of the invention.

FIG. 10 is a flowchart illustrating the method of removing offensive odors from ambient air in accordance with an implementation of the invention.

FIG. 11A-11C are various views illustrating the movement of ambient air through the odor detection and filtration device in accordance with an implementation of the invention.

FIG. 12 shows an exemplary electrical circuit of the components of the odor detection and filtration device in accordance with an implementation of the invention.

### DETAILED DESCRIPTION OF THE INVENTION

Implementations of the invention provide an odor detection and filtration device for removing odors resulting from flatulence, defecation, or any unpleasant or offensive odors.

The odor detection and filtration device may be used in various locations to improve the condition of ambient air surrounding the device by detecting and removing gases commonly found in odors resulting from flatulence or released during defecation and/or urination in both humans and domestic animals. For example, the device may be placed under the sheets or covers of a bed, in the vicinity of a bed, in a bathroom, an office, automobile, bus, airplane, or any location that would be susceptible to gases commonly found in odors resulting from flatulence or released during defecation and/or urination in both humans and domestic animals.

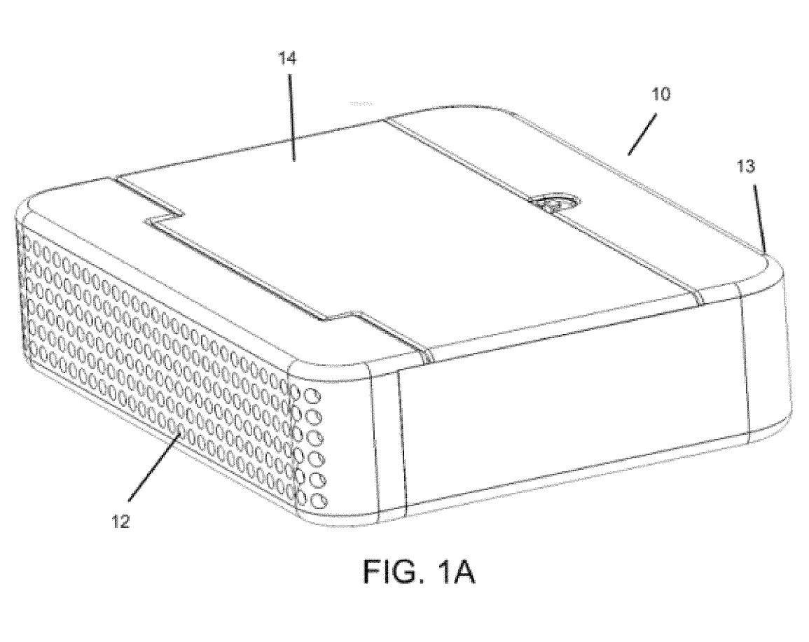

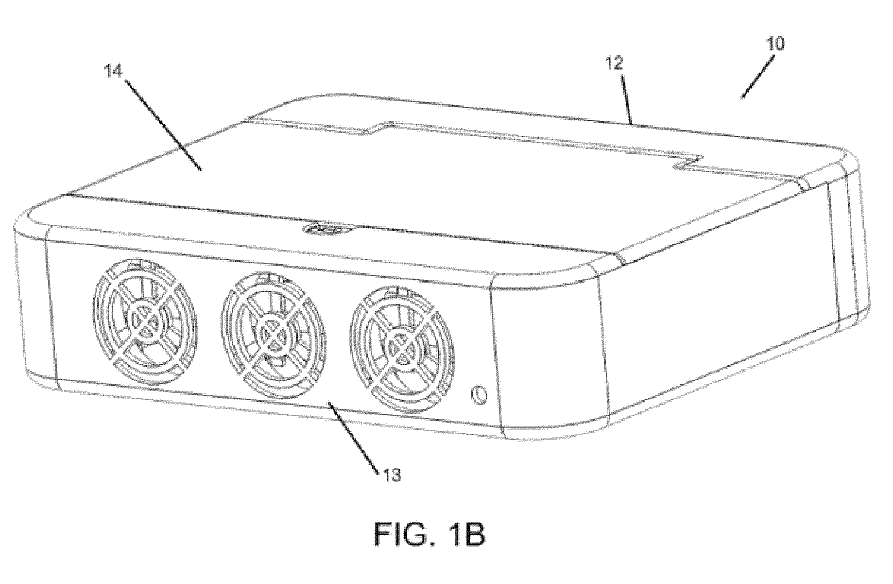

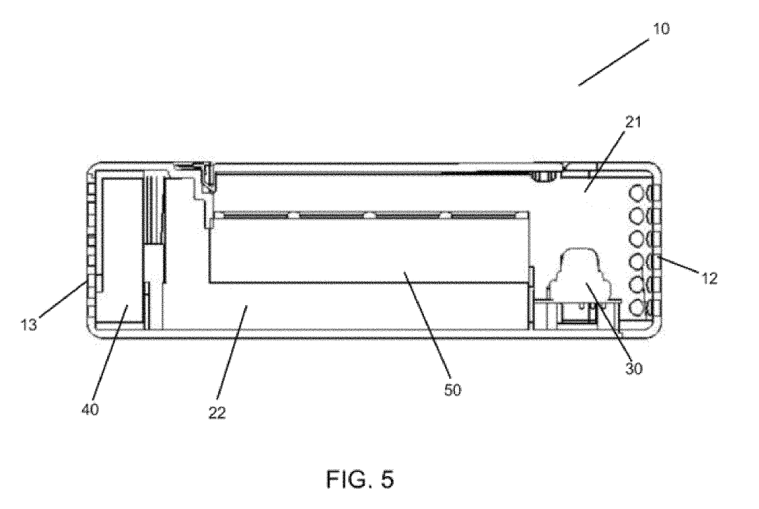

Referring to FIGS. 1A-1B, an example implementation of an odor detection and filtration device 10 is shown including a generally hollow housing 11 having an air inlet 12, an air outlet 13, and an access cover 14. The frame of the housing 11 may be substantially rectangular; however the housing 11 is not limited to any particular shape, and may be rectangular, square, round, octagonal, trapezoidal, hexagonal or oval, among other shapes. The housing 11 may be constructed of a rigid material such as metal, plastic, glass, or the like. The housing 11 may be aesthetically appealing to users and may be integrated into the interior or exterior of constructed items or furniture such as beds, seats, chairs, couches, desks, or other structures where individuals sit, rest, or work. The generally hollow interior of the housing 11 defines an open space that may be referred to as the “void” (described in further detail below with respect to FIG. 5). The void facilitates the collection of ambient air that enters the housing 11 and defines an air passage that extends from the air inlet 12 to the air outlet 13.

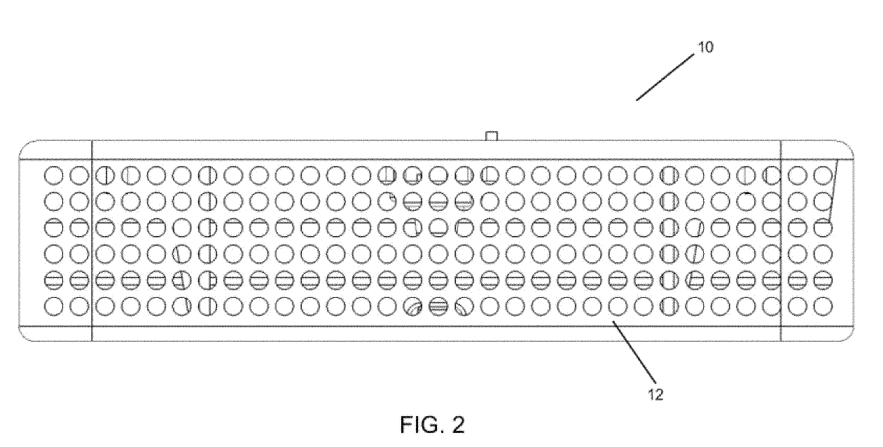

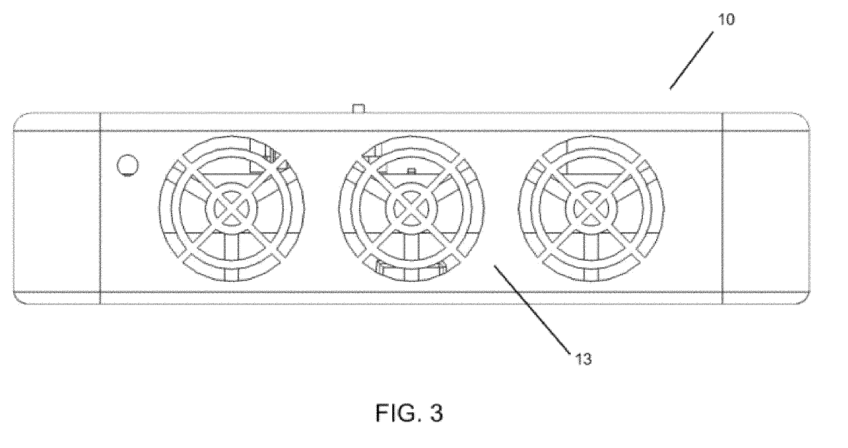

FIG. 2 illustrates the air inlet 12 located at the periphery of the housing 11. The air inlet 12 may have a plurality of apertures to allow air from outside the housing 11 to enter the void. FIG. 3 shows the air outlet 13 located at the periphery of the housing 11. The air outlet 13 is positioned opposite from the air inlet 12. The air outlet 13 may have a plurality of apertures to allow collected air to evacuate from the void to outside the housing 11.

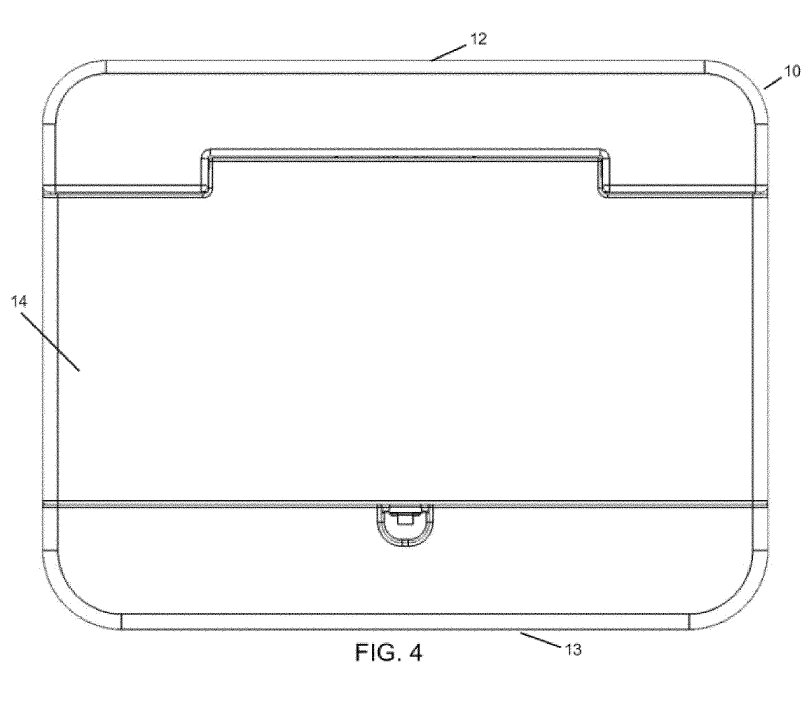

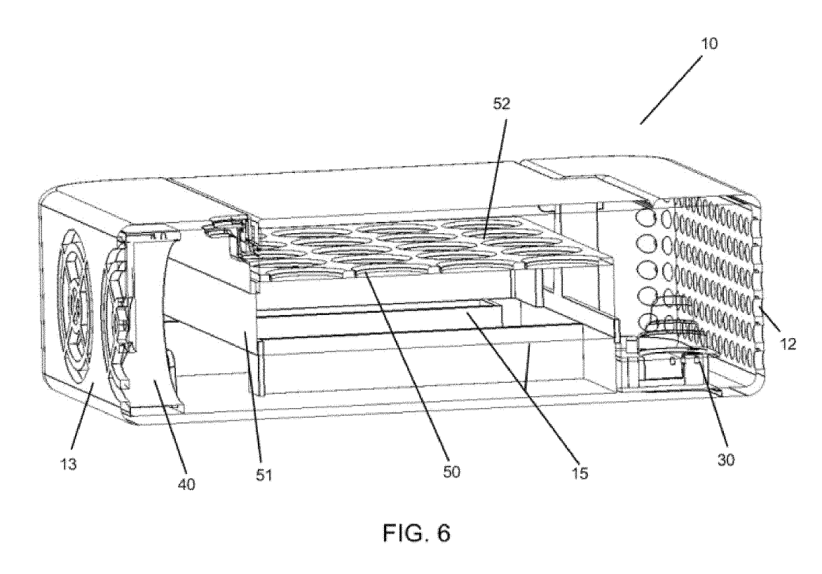

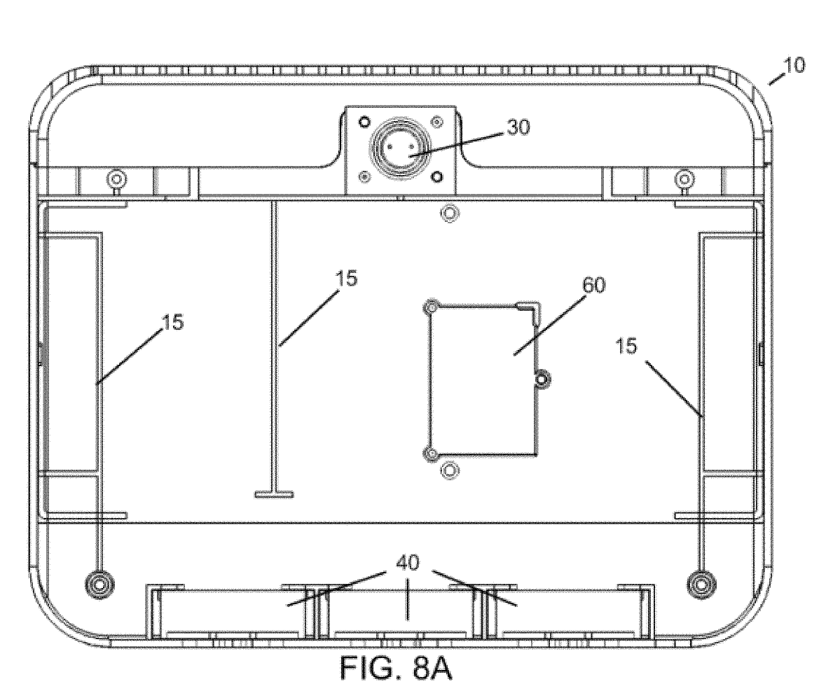

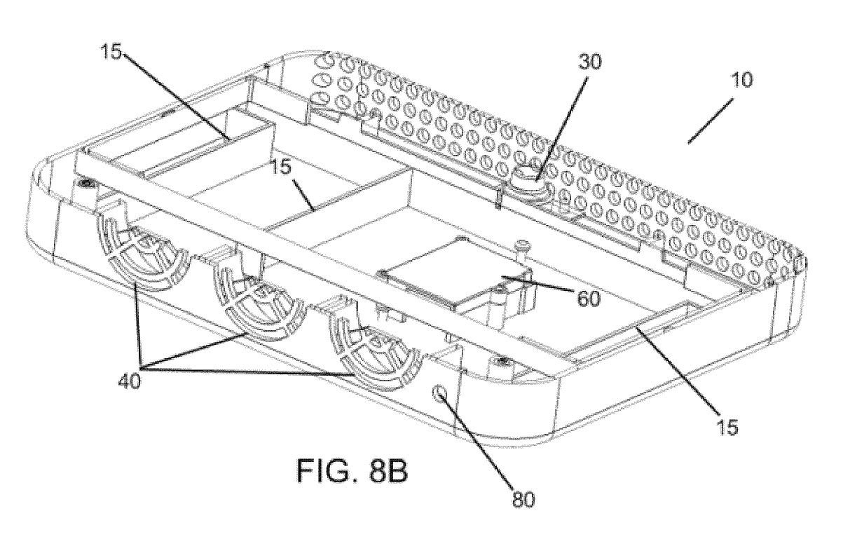

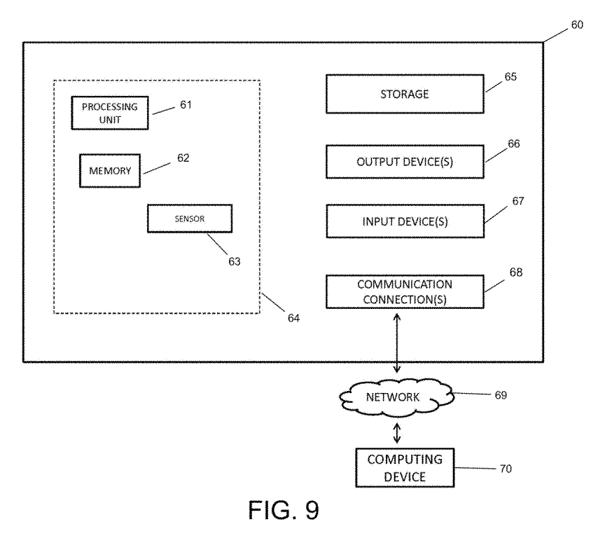

Referring to FIG. 4, the housing 11 may include an access cover 14 to allow a user to access the void and remove or replace a filter (described in further detail below with respect to FIGS. 6 & 7). The access cover 14 may be fully removable or may actuate about a hinge positioned within the frame of the housing 11. In another implementation, the housing 11, or specifically the access cover 14, may include a display, for example an LED screen, an LCD screen, or another interactive device that communicates with a control unit (described in further detail below with respect to FIGS. 8A, 8B, and 9) to control various settings and functions of the device 10. In a further implementation, the housing 11 of the odor detection and filtration device 10 may include various knobs, buttons, or indication lights that may correspond to various settings or functions of the device 10.

FIG. 5 shows a cross section side view of the odor detection and filtration device 10 illustrating the interior of the housing 11. The generally hollow interior of the housing 11 defines an open space that may be referred to as the void. The void facilitates the collection of air that enters the housing 11 and defines an air passage that extends from the air inlet 12 to the air outlet 13. In one implementation, the void may include one or more chambers. Further, in the example implementation shown in FIG. 5, the void may be comprised of an upper chamber 21 and a lower chamber 22, and one or more filters 50 may form an intermediary between the upper chamber 21 and the lower chamber 22.

The void contains, and the housing 11 encloses, one or more sensors 30, one or more filters 50, one or more fans 40, and a control unit. In a further implementation, a power supply may be included within the housing 11; however, the power supply may be external (described in greater detail below with respect to FIGS. 8A & 8B).

FIGS. 6 & 7 show a cross section side angle view and a cross section top view, respectively, that illustrates a sensor 30, a filter 50, and one or more fans 40, in accordance with an implementation of the odor detection and filtration device 10.

Referring to FIGS. 6 & 7, the odor detection and filtration device 10 includes one or more electrical, optical, chemical or bioengineered sensors 30 that detect the presence of one or more gases or chemical compounds commonly found in, or associated with, odors resulting from flatulence or released during defecation and/or urination in both humans and domestic animals. The one or more gases include, but are not limited to, hydrogen (H2), methane, indole, skatole, ammonia (NH3), mercaptans, hydrogen sulfide (HS2), methyl mercaptan, MM (also known as methanethiol, MT), dimethyl sulfide (DMS), dimethyl disulfide (DMDS), dimethyl trisulfide (DMTS), and other volatile sulfur compounds (VSC).

The one or more sensors 30 may be electrical, optical, chemical or bioengineered, or any other suitable type. Non limiting examples of known sensors that could be used include:

MOS-Type

MQ-4—Methane Gas Sensor

MQ8—Hydrogen Sensor

MQ9—Flammable Gas Sensor

MQ136—H₂S (Hydrogen Sulfide) Sensor

MQ135—Hazardous Gas Sensor

MQ137—Ammonia Sensor

MQ138—Multiple Gas Sensor

MQ309A—Flammable Gas Sensor

AQ104—Air Quality Sensor

MiCS-2714—Multiple Gas Sensor

MP901—Air-Quality Gas Sensor VOC Gas Sensor

MP905—Air-Quality Gas Sensor

Electrochemical-Type

ME3—N₂ Electrochemical Hydrogen Gas Sensor

ME3—NH₃ Electrochemical Ammonia Sensor

ZE03—Electrochemical Gas Sensor Module

ZE12—Electrochemical Gas Sensor Module For Atmospheric Monitoring

ME3—H₂S Hydrogen Sulfide Sensor

Methanethiol Sensor (e.g. 4-Series CH3SH-10 by Semeatech)

Constant Potential Electrolysis-Type

ME4—H₂ Hydrogen Gas Sensor—H2

ME4—NH₃ Ammonia Gas Sensor

ME4—H₂S Hydrogen Sulfide Gas Sensor

MEMS-Type

GM-702B—MEMS Carbon Monoxide Gas Sensor

GM-402B—MEMS Combustible Gas Sensor

GM-502B—MEMS VOC Gas Sensor

GM-602B—MEMS H₂S Hydrogen Sulfide Gas Sensor

Catalytic Type

MC101—Catalytic Flammable Gas Sensor/Methane Sensor

Infrared-Type

MH-741A—Infrared Gas Sensor/NDIR Combustible Gas Detection/C₃H₈ Sensing includes CH₄ and possibly H₂

Explosive Gas Infrared Sensors (models vary)

H₂S Infrared Sensors (models vary)

In an example implementation, an MOS-type sensor may be the most economical sensor type for detection of hydrogen. Preferably, one or more compact sensors of any of the types described above are used to mitigate the exhaustion of space within the void of the odor detection and filtration device 10. In a preferred implementation, the one or more sensors 30 are positioned near the air inlet 12 of the device 10 to easily detect the composition of ambient air.

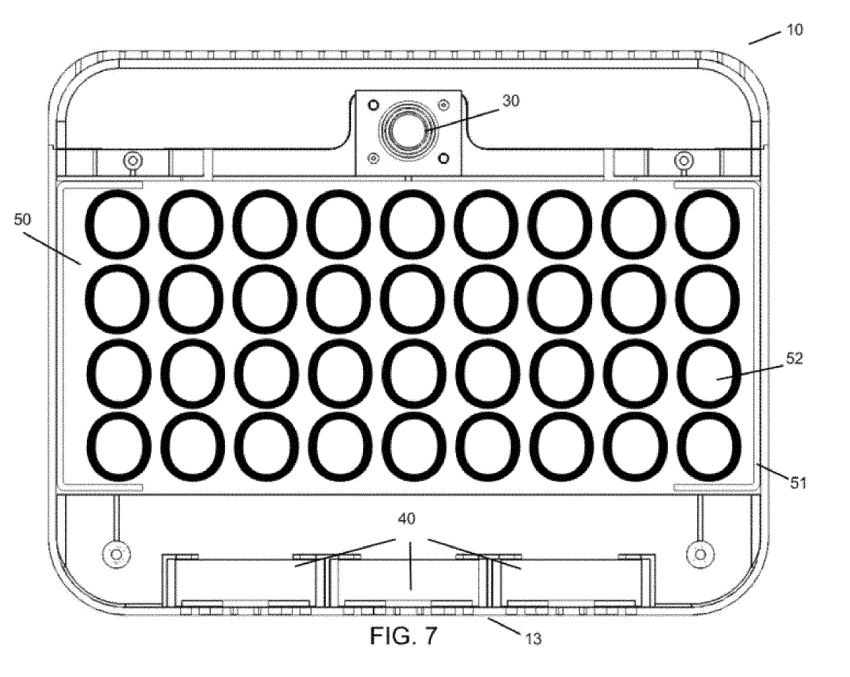

The filter 50 is disposed within the void of the housing 11 and is adapted to remove odors and particulates from ambient air that has entered the void. As described in the example implementation above with respect to FIG. 5, the filter 50 may form an intermediary between the upper chamber (21) and the lower chamber (22) of the void. The filter 50 may be constructed with a substantially rigid frame 51 that supports and encloses the filter 50. The frame 51 of the filter 50 may have a plurality of holes 15 to allow ambient air to travel through the frame 51 of the filter 50 and the filter 50 itself. The frame 51 of the filter 50 may be seated on one or more brackets 15 (not shown in FIG. 7, but refer to FIGS. 8A & 8B for a top view of the device 10 that shows the brackets) within the housing 11 to maintain the positioning of the filter 50. The filter 50 may be removable and replaceable. The filter 50 may optionally be washed or vacuumed to clean the filter from contaminants.

The filter 50 may be comprised of a receptacle of a variety of shapes and materials containing one or more odor absorbent materials such as activated carbon in granules, powder or other variety of physical forms, or any other odor absorbing materials such as bismuth subgallate that is capable of absorbing and removing gases that are found in odors resulting from flatulence, feces, and urine. In addition, the filter 50 may be capable of removing other airborne contaminants such as dust, pollen, mold spores, pet dander and the like. In another implementation, the filter 50 may be a high efficiency particulate (HEPA) filter of the type which has been widely utilized in the medical, healthcare, and pharmaceutical fields to entrap airborne particulates in the submicron range. In a further implementation, one or more filters may be used to provide multiple stage treatment of ambient air.

In another implementation, the odor detection and filtration device 10 may lack the presence of a filter 50, and instead transport the one or more odors away from the vicinity in which the device 10 and user are located. The one or more odors may be transported via a duct positioned within the housing and adapted to expel the one or more odors, or via the air outlet 13.

Still referring to FIGS. 6 & 7, the one or more fans 40 may be positioned at the air outlet 13 of the device 10 and adapted to draw ambient air through the air inlet 12 into the void and subsequently downstream through the filter 50 and out the device 10 through the air outlet 13. In a preferred implementation, the device 10 has at least three fans 40 to provide sufficient suction power to advance the ambient air through the air inlet 12 into the void and subsequently downstream through the filter 50 and out the device 10 through the air outlet 13. The one or more fans 40 can be activated or deactivated upon the one or more sensors 30 detecting the presence or absence of one or more odors in the ambient air surrounding the device 10. The one or more fans 40 may have variable speeds and variable durations of activation that may be controlled by the control unit (60) (described below with respect to FIGS. 8A & 8B).

In one implementation, the odor filtration device can include a pleasant odor mechanism that releases a fragrance that is pleasing to humans into the ambient air. The fragrance may be released simultaneously or subsequently with the activation of the one or more fans 40, or deactivation of one or more fans 40. The fragrance may be released into the ambient air surrounding the housing 11 upon the one or more sensors 30 detecting the presence of one or more odors in the ambient air surrounding the housing 11.

In another implementation, the odor filtration device can include an ultraviolet (UV) light. The UV light is adapted to sterilize substantially all of the airborne micro-organisms (for example, bacteria, viruses, germs, and mold spores) contained in the ambient air entering the void through the air inlet 12 prior to passing through the filter 50 or after passing through the filter 50. The UV light can be positioned in the void in the upper chamber (21) or lower chamber (22) (as described above with respect to FIG. 5) at the air inlet 12 or air outlet 13. The UV light may be oriented to maximize the UV light's exposure to ambient air that is passed through the void. The UV light is removable and replaceable. The UV light may be manually activated by a user of the device 10, or the UV light may be activated automatically when the one or more fans 40 are activated or it can be independently activated by the control unit (60). The housing of the device 10 is constructed to prevent the passage of damaging UV light rays to outside the device 10.

FIGS. 8A & 8B are cross section top and cross section top angle views, respectively, of the odor detection and filtration device 10 that illustrate the control unit 60 positioned within the void of the device 10. The control unit 60 may be in electrical communication with the one or more sensors 30 and one or more fans 40. As described above, the control unit 60 may activate the one or more fans 40 upon the one or more sensors 30 detecting the presence of one or more odors in the ambient air surrounding the device 10. Further, the control unit 60 may deactivate the one or more fans 40 upon the one or more sensors 30 detecting the absence of one or more odors in the ambient air surrounding the device 10. In another implementation, the control unit 60 may be in further electrical communication with the ultraviolet (UV) light described above. The control unit 60 may activate the UV light upon the one or more fans 40 activating due to the one or more sensors 30 detecting the presence of one or more odors in the ambient air surrounding the device 10. Further, the control unit 60 may deactivate the UV light upon the one or more fans 40 deactivating due to the one or more sensors 30 detecting the absence of one or more odors in the ambient air surrounding the housing 11.

FIG. 9 and the following discussion provide a brief, general description of a suitable computing environment to implement implementations of one or more of the provisions set forth herein. The operating environment of FIG. 9 is only one example of a suitable operating environment and is not intended to suggest any limitation as to the scope of use or functionality of the operating environment. Example computing devices include, but are not limited to, personal computers, server computers, hand-held or laptop devices, mobile devices (such as mobile phones, Personal Digital Assistants (PDAs), media players, and the like), multiprocessor systems, consumer electronics, mini computers, mainframe computers, embedded systems, distributed computing environments that include any of the above systems or devices, and the like.

Although not required, implementations are described in the general context of “computer readable instructions” being executed by one or more computing devices. Computer readable instructions may be distributed via computer readable media (discussed below). Computer readable instructions may be implemented as program modules, such as functions, objects, Application Programming Interfaces (APIs), data structures, and the like, that perform particular tasks or implement particular abstract data types. Typically, the functionality of the computer readable instructions may be combined or distributed as desired in various environments.

FIG. 9 illustrates an example of an odor detection and filtration device comprising a control unit 60 configured to implement one or more implementations provided herein. In one implementation, the control unit 60 includes at least one processing unit 61 at least one memory 62, and optionally at least one sensor 63. Depending on the exact configuration and type of control unit 60, memory 62 may be volatile (such as RAM, for example), non-volatile (such as ROM, flash memory, etc., for example) or some combination of the two. This configuration is illustrated in FIG. 9 by dashed line 64.

In other implementations, control unit 60 may include additional features and/or functionality. For example, control unit 60 may also include additional storage (e.g., removable and/or non-removable) including, but not limited to, magnetic storage, optical storage, and the like. Such additional storage is illustrated in FIG. 9 by storage 65. In one implementation, computer readable instructions used to implement one or more implementations provided herein may be in storage 65. Storage 65 may also store other computer readable instructions to implement an operating system, an application program, and the like. Computer readable instructions may be loaded in memory 62 for execution by processing unit 61, for example.

The term “computer readable media” as used herein includes computer storage media. Computer storage media includes volatile and nonvolatile, removable and non-removable media implemented in any method or technology for storage of information such as computer readable instructions or other data. Memory 62 and storage 65 are examples of computer storage media. Computer storage media includes, but is not limited to, RAM, ROM, EEPROM, flash memory or other memory technology, CD-ROM, Digital Versatile Disks (DVDs) or other optical storage, magnetic cassettes, magnetic tape, magnetic disk storage or other magnetic storage devices, or any other medium which can be used to store the desired information and which can be accessed by control unit 60. Any such computer storage media may be part of control unit 60.

Control unit 60 may also include communication connection(s) 68 that allows control unit 60 to communicate with other devices. Communication connection(s) 68 may include, but is not limited to, a modem, a Network Interface Card (NIC), an integrated network interface, a radio frequency transmitter/receiver, an infrared port, a USB connection, or other interfaces for connecting control unit 60 to other computing devices. Communication connection(s) 68 may include a wired connection or a wireless connection. Communication connection(s) 68 may transmit and/or receive communication media.

The term “computer readable media” may include communication media. Communication media typically embodies computer readable instructions or other data in a “modulated data signal” such as a carrier wave or other transport mechanism and includes any information delivery media. The term “modulated data signal” may include a signal that has one or more of its characteristics set or changed in such a manner as to encode information in the signal.

Control unit 60 may include input device(s) 67 such as keyboard, mouse, pen, voice input device, touch input device, infrared cameras, video input devices, and/or any other input device. Output device(s) 66 such as one or more displays, speakers, printers, and/or any other output device may also be included in control unit 60. Input device(s) 67 and output device(s) 66 may be connected to control unit 60 via a wired connection, wireless connection, or any combination thereof. In one implementation, an input device or an output device from another computing device may be used as input device(s) 67 or output device(s) 66 for control unit 60.

Components of control unit 60 may be connected by various interconnects, such as a bus, like, for example, an NMEA2000 Can Bus. Such interconnects may include a Peripheral Component Interconnect (PCI), such as PCI Express, a Universal Serial Bus (USB), firewire (IEEE 1394), an optical bus structure, and the like. In another implementation, components of control unit 60 may be interconnected by a network. For example, memory 62 may be comprised of multiple physical memory units located in different physical locations interconnected by a network.

Those skilled in the art will realize that storage devices utilized to store computer readable instructions may be distributed across a network. For example, a computing device 70 accessible via network 69 may store computer readable instructions to implement one or more implementations provided herein. Control unit 60 may access computing device 70 and download a part or all of the computer readable instructions for execution. Alternatively, computing device 70 may download pieces of the computer readable instructions, as needed, or some instructions may be executed at control unit 60 and some at computing device 70.

The control unit 60 can manage and influence settings and functions including, but not limited to, the length of time the one or more fans operate, the duration of time the device is operation, the time between filter changes, the detection of specific odors, the activation or deactivation of the UV light, the activation or deactivation of the pleasant odor-producing mechanism, an indication to replace the filter, and an indication to replace the UV light. As described above with respect to FIG. 9, a user may manually operate the odor detection and filtration device and manage and influence settings and functions via an input device such as an LED screen, LCD screen, computerized touch screen, or any similar interactive device located on the device, or via a Bluetooth or other wireless connection to a smartphone, tablet, or similar computerized device that includes a computer application that communicated with the control unit 60 via the wireless connection. In a further implementation, a user may manually operate the odor detection and filtration device by adjusting various knobs or buttons located on the odor detection and filtration device that may correspond to various settings or functions of the odor detection or filtration device.

Referring back to FIGS. 8A & 8B, the odor detection and filtration device 10 also includes a power supply (not shown) adapted to supply power to the device 10. More specifically, the power supply supplies power to the one or more fans 40, one or more sensors 30, and control unit 60. In a further implementation, the power supply can supply power to the UV light or pleasant odor-producing mechanism. The power supply may be in the form of one or more batteries that are replaceable and/or rechargeable. In another implementation, as shown in FIG. 8B, an electric cord port may be included in the housing of the device to allow an electrical power cable attached to an external power source to power the device 10 and the one or more fans 40, one or more sensors 30, control unit 60, UV light, or pleasant odor-producing mechanism.

In an alternative implementation of the present invention (not shown) an enclosed housing (and therefore air inlet and air outlet) are not utilized. Instead, the sensors, fans, filters, power supplies, UV lights and control unit are arranged and secured to a frame. The fan(s) and filter(s) in such an implementation would be linearly arranged so that air being drawn or pushed by the fan(s) is forced through the filter(s) and/or the UV lights. The operation of such an alternative implementation would otherwise be unchanged from that previously described.

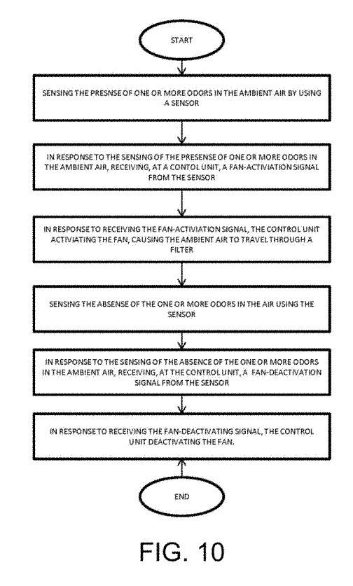

FIG. 10 illustrates an exemplary method for removing offensive odors from ambient air in accordance with the implementations of the odor detection and filtration device as described above. The method begins when one or more sensors sense the presence of one or more odors in the ambient air. In response to sensing the presence of one or more odors in the ambient air, the one or more sensors send a fan-activation signal to the control unit. In response to the control unit receiving the fan-activation signal, the control unit activates one or more fans, which causes the ambient air to travel through a filter. While the one or more fans cause the ambient air to travel through the filter, the one or more sensors continue to sense for the presence of one or more odors. When the one or more sensors sense the absence of the one or more odors that were originally detected in the ambient air, the one or more sensors send a fan-deactivation signal to the control unit. In response to the control unit receiving the fan-deactivation signal, the control unit deactivates the one or more fans.

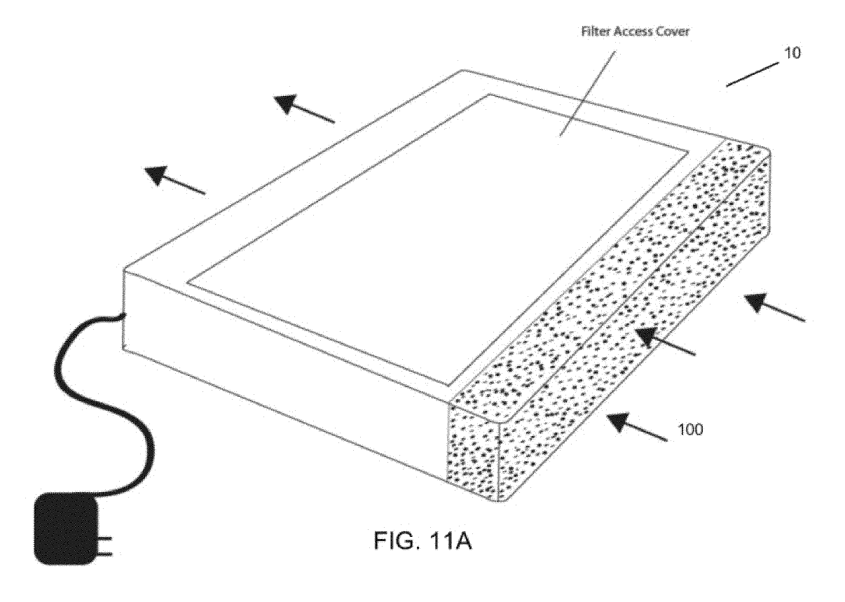

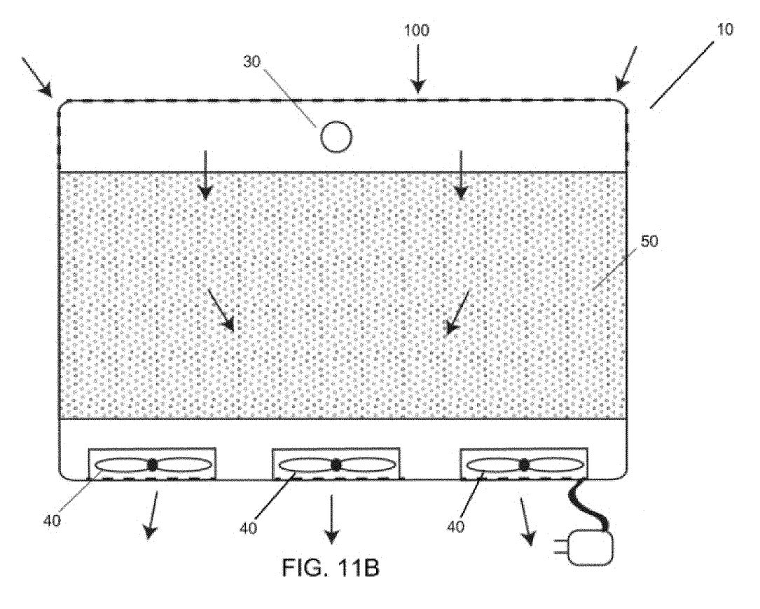

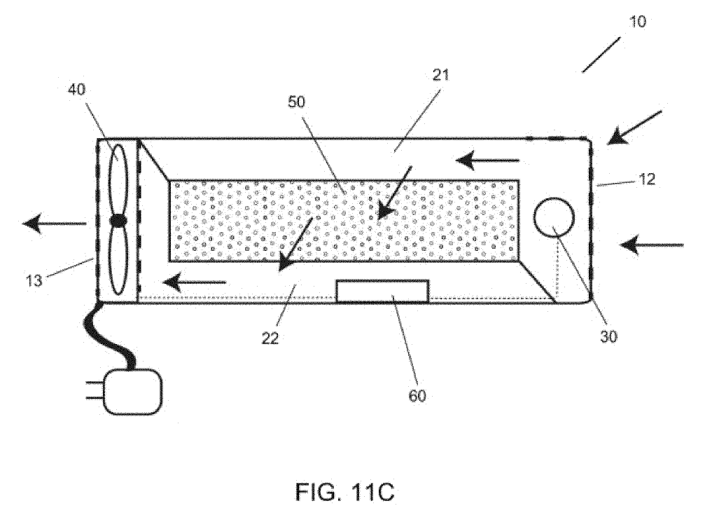

FIGS. 11A-11C illustrate the movement of ambient air through the housing of the odor detection and filtration device in accordance with the example implementation of the method described above with respect to FIG. 10. In FIG. 11A-11C, arrows 100 represent the movement of ambient air (100) through the device. In response to the one or more sensors 30 sensing the presence of the one or more odors in the ambient air (100), the one or more sensors 30 send a fan-activation signal to the control unit 60. In response to receiving the fan-activation signal, the control unit 60 activates one or more fans 40, causing the device 10 to draw ambient air (100) through the air inlet 12 into the void and subsequently downstream through the filter 50 and out the device 10 through the air outlet 13.

Referring specifically to FIGS. 11B and 11C, as the ambient air (100) enters the air inlet 12, the filter 50 positioned within the void of the housing treats the ambient air (100) by removing the one or more odors as the one or more fans 40 draws the ambient air (100) into the void. The ambient air (100) travels through the upper chamber 21 (shown in FIG. 11C) of the void and moves downstream through the filter 50 to subsequently travel through the lower chamber 22 (shown in FIG. 11C) of the void and exit the device 10 through the air outlet 13. Upon passing through the filter 50, the ambient air (100) exits the void with the one or more odors removed.

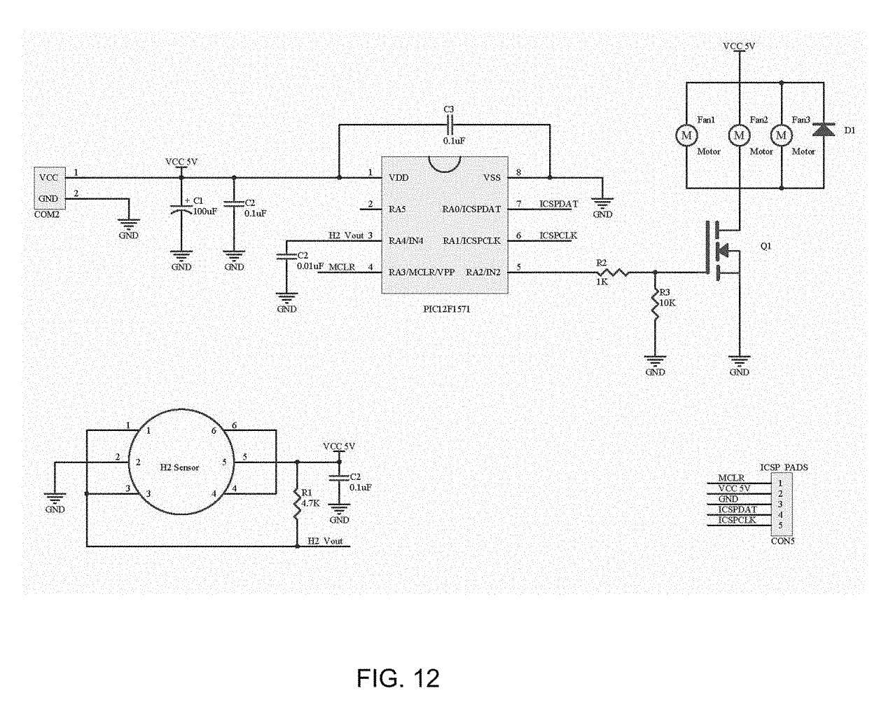

FIG. 12 shows an exemplary electrical circuit that illustrates the distribution of power among the various elements of the odor detection and filtration device in accordance with previously described implementations of the device. This exemplary electrical circuit is for illustrative purposes and should therefore not limit the invention or any implementation of the invention. The exemplary electrical circuit will be appreciated by one skilled in the art having the benefit of this illustration.

Any reference in this specification to “one implementation,” “an implementation,” an “example implementation,” etc., means that a particular feature, structure, or characteristic described in connection with the implementation is included in at least one implementation of the invention. The appearances of such phrases in various places in the specification are not necessarily referring to the same implementation. In addition, any elements or limitations of any invention or implementation thereof disclosed herein can be combined with any and/or all other elements or limitations (individually or in any combination) or any invention or implementation thereof disclosed herein, and all such combinations are contemplated with the scope of the invention without limitation thereto.

It should be understood that the examples and implementations described herein are for illustrative purposes only and that various modifications or changes in light thereof will be suggested to persons skilled in the art and are to be included within the spirit and purview of this application.

We claim:

1. An odor filtration device, comprising:
a housing enclosing a void and having an air inlet and an air outlet, the air inlet adapted to receive ambient air from outside the housing into the void, and the air outlet adapted to evacuate ambient air from the void to outside the housing;
one or more sensors adapted to detect the presence of one or more gasses or chemical compounds associated with odors in the ambient air surrounding the housing;
one or more fans adapted to draw ambient air into the void;
a control unit in communication with the one or more sensors and the one or more fans, wherein the control unit activates the one or more fans upon the one or more sensors detecting the presence of the one or more odors in the ambient air surrounding the housing;
a filter disposed within the void and adapted to remove odors from ambient air entering the void; and
a power supply adapted to supply power to the one or more fans, the one or more sensors, and the control unit.

2. The odor filtration device of claim 1 further comprising an input device adapted to enable a user to manually operate the control unit to activate or deactivate the one or more fans.

3. The odor filtration device of claim 2, wherein the input device wirelessly communicates with the control unit.

4. The odor filtration device of claim 1 further comprising an ultraviolet light adapted to sterilize the ambient air entering the void through the air inlet.

5. The odor filtration device of claim 1 further comprising a fragrance that is released into the ambient air surrounding the housing upon the sensor detecting the presence of one or more gasses or chemical compounds associated with odors in the ambient air surrounding the housing.

6. The odor filtration device of claim 1, wherein the air inlet has a plurality of apertures.

7. The odor filtration device of claim 1, wherein the air outlet has a plurality of apertures.

8. The odor filtration device of claim 1, wherein the void is comprised of an upper chamber and a lower chamber.

9. The odor filtration device of claim 1, wherein the one or more sensors are selected from the group of sensor types consisting of MOS, electrochemical, constant potential electrolysis, MEMS, catalytic and infrared.

10. The odor filtration device of claim 1, wherein the power supply is one or more batteries or an external power supply.

11. The odor filtration device of claim 1, wherein the housing further includes an access cover to allow the filter to be removed and replaced.

12. The odor filtration device of claim 1, wherein the filter is a HEPA filter.

13. A method for removing offensive odors from ambient air, the method comprising:
sensing the presence of one or more one or more gasses or chemical compounds associated with odors in the ambient air by using one or more sensors;
in response to the sensing of the presence of the one or more odors in the ambient air, receiving, at a control unit, a fan-activation signal from the one or more sensors;
in response to receiving the fan-activation signal, the control unit activating one or more fans, causing the ambient air to travel through a filter;
sensing the absence of the one or more odors in the ambient air using the one or more sensors;
in response to the sensing of the absence of the one or more odors in the ambient air, receiving, at the control unit, a fan-deactivation signal from the sensor; and
in response to receiving the fan-deactivating signal, the control unit deactivating the one or more fans.

14. The method of claim 13 further comprising activating an ultraviolet light adapted to sterilize the ambient air upon the one or more fans receiving the fan-activation signal.

15. The method of claim 13 further comprising releasing a fragrance into the ambient air surrounding the housing upon the sensor detecting the presence of one or more gasses or chemical compounds associated with odors in the ambient air.

16. The method of claim 13, wherein the one or more sensors are selected from the group of sensor types consisting of MOS, electrochemical, constant potential electrolysis, MEMS, catalytic and infrared.

17. A method for removing offensive odors from ambient air, the method comprising:
providing an odor filtration device, comprising: a housing enclosing a void and having an air inlet and an air outlet, the air inlet adapted to receive ambient air from outside the housing into the void, and the air outlet adapted to evacuate ambient air from the void to outside the housing; one or more sensors adapted to detect the presence of one or more gasses or chemical compounds associated with odors in the ambient air surrounding the housing; one or more fans adapted to draw ambient air into the void; a control unit in communication with the one or more sensors and the one or more fans; a filter disposed within the void and adapted to remove odors from ambient air entering the void; and a power supply adapted to supply power to the one or more fans, the one or more sensors, and the control unit;
sensing the presence of one or more gasses or chemical compounds associated with odors in the ambient air by using the one or more sensors;
in response to sensing the presence of the one or more odors in the ambient air, receiving, at the control unit, a fan-activation signal from the one or more sensors;
in response to receiving the fan-activation signal, the control unit activating the one or more fans, causing the ambient air to travel through the filter;
sensing the absence of the one or more odors in the ambient air using the one or more sensors;
in response to the sensing of the absence of the one or more odors in the ambient air, receiving, at the control unit, a fan-deactivation signal from the one or more sensors; and
in response to receiving the fan-deactivating signal, the control unit deactivating the one or more fans.

18. The method of claim 17 further comprising activating an ultraviolet light adapted to sterilize the ambient air upon the one or more fans receiving the fan-activation signal.

19. The method of claim 17 further comprising releasing a fragrance into the ambient air surrounding the housing upon the sensor detecting the presence of one or more gasses or chemical compounds associated with odors in the ambient air.

20. The method of claim 17, wherein the one or more sensors are selected from the group of sensor types consisting of MOS, electrochemical, constant potential electrolysis, MEMS, catalytic and infrared.

21. An odor filtration device, comprising:
a frame;
one or more sensors adapted to detect the presence of one or more gasses or chemical compounds associated with odors in the ambient air surrounding the frame;
one or more fans adapted to draw ambient air to the frame;
a control unit in communication with the one or more sensors and the one or more fans, wherein the control unit activates the one or more fans upon the one or more sensors detecting the presence of the one or more odors in the ambient air surrounding the housing;
a filter disposed on the frame and adapted to remove odors from ambient air drawn by the one or more fans; and
a power supply adapted to supply power to the one or more fans, the one or more sensors, and the control unit.

22. The odor filtration device of claim 21 further comprising an input device adapted to enable a user to manually operate the control unit to activate or deactivate the one or more fans.

23. The odor filtration device of claim 21, wherein the input device wirelessly communicates with the control unit.

24. The odor filtration device of claim 21 further comprising an ultraviolet light adapted to sterilize the ambient air drawn to the frame.

25. The odor filtration device of claim 21 further comprising a fragrance that is released into the ambient air surrounding the housing upon the one or more sensors detecting the presence of one or more gasses or chemical compounds associated with odors in the ambient air surrounding the housing.

26. The odor filtration device of claim 21, wherein the frame has a plurality of apertures.

27. The odor filtration device of claim 21, wherein the one or more sensors are selected from the group of sensor types consisting of MOS, electrochemical, constant potential electrolysis, MEMS, catalytic and infrared.

28. The odor filtration device of claim 21, wherein the power supply is one or more batteries or an external power supply.

29. The odor filtration device of claim 21, wherein the filter is adapted to be removed and replaced.

30. The odor filtration device of claim 21, wherein the filter is a HEPA filter.
